(19) United States
(12) Patent Application Publication
(10) Pub. No.: US 2009/0281133 A1
(43) Pub. Date: Nov. 12, 2009

(54) Heterocyclic antiviral compounds

(75) Inventors: David Mark Rotstein, Sunnyvale, CA (US); Hanbiao Yang, Sunnyvale, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 12/387,921

(22) Filed: May 8, 2009

Related U.S. Application Data
(60) Provisional application No. 61/051,743, filed on May 9, 2008.

(51) Int. Cl. A61K 31/438; C07D 215/02; A61P 31/18

(52) U.S. Cl. 514/278; 546/16

(57) ABSTRACT

This invention relates to piperidine derivatives of formula I wherein R¹, R² and R³ are as defined herein useful in the treatment of a variety of disorders, including those in which the modulation of CCR5 receptors is implicated. Disorders that may be treated or prevented by the present derivatives include HIV and genetically related retroviral infections (and the resulting acquired immune deficiency syndrome, AIDS), rheumatoid arthritis, solid organ transplant reject (graft vs. host disease), asthma and COPR.

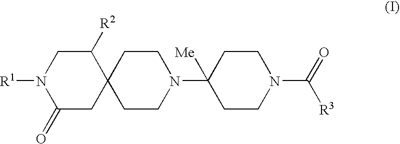

### CROSS REFERENCE TO PRIOR APPLICATIONS

This application claims the benefit of priority to U.S. Ser. No. 61/051,743 filed May 9, 2008 which is hereby incorporated in its entirety by reference.

### FIELD OF THE INVENTION

This invention relates to 3,9-diaza-spiro[5.5]undecan-2-one compounds useful for the treatment of a variety of disorders in which modulation of the CCR5 receptor ligand binding is beneficial. More particularly, to new and 9-(4-methyl-piperidin-4-yl)-3,9-diaza-spiro[5.5]undecan-2-one compounds, to compositions containing said compounds and to uses of such compounds and compositions. Disorders that may be treated or prevented by the present compounds include HIV-1 (and the resulting acquired immune deficiency syndrome, AIDS), arthritis, asthma, chronic obstructive pulmonary disease (COPD) and rejection of transplanted organs.

### BACKGROUND OF THE INVENTION

Compounds of the present invention modulate the activity of the chemokine CCR5 receptors. The CCR5 receptor is a member of a subset of a large family chemokine receptors characterized structurally by two adjacent cysteine residues. Human chemokines include approximately 50 small proteins of 50-120 amino acids that are structurally homologous. (M. Baggiolini et al., Ann. Rev. Immunol. 1997 15:675-705) The chemokines are pro-inflammatory peptides that are released by a wide variety of cells such as macrophages, monocytes, eosinophils, neutrophiles, fibroblasts, vascular endothelial cells, smooth muscle cells, and mast cells, at inflammatory sites (reviewed in Luster, New Eng. J Med. 1998 338:436-445 and Rollins, Blood 1997 90:909-928). The name “chemokine”, is a contraction of “chemotactic cytokines”. The chemokines are a family of leukocyte chemotactic proteins capable of attracting leukocytes to various tissues, which is an essential response to inflammation and infection. Chemokines can be grouped into two subfamilies, based on whether the two amino terminal cysteine residues are immediately adjacent (CC family) or separated by one amino acid (CXC family). The CXC chemokines, such as interleukin-8 (IL-8), neutrophil-activating protein-2 (NAP-2) and melanoma growth stimulatory activity protein (MGSA) are chemotactic primarily for neutrophils and T lymphocytes, whereas the CC chemokines, such as RANTES (CCL5), MIP-1α (CCL3, macrophage inflammatory protein), MIP-1β (CCL4), the monocyte chemotactic proteins (MCP-1, MCP-2, MCP-3, MCP-4, and MCP-5) and the eotaxins (−1 and −2) are chemotactic for, among other cell types, macrophages, T lymphocytes, eosinophils, dendritic cells, and basophils. Naturally occurring chemokines that can stimulate the CCR5 receptor include MIP-1α, MIP-1β and RANTES.

Accordingly, drugs which inhibit the binding of chemokines such as MIP-1α, MIP-1β and RANTES to these receptors, e.g., chemokine receptor antagonists, may be useful as pharmaceutical agents which inhibit the action of chemokines such as MIP-1α, MIP-1β and RANTES on the target cells. The identification of compounds that modulate the function of CCR5 represents an excellent drug design approach to the development of pharmacological agents for the treatment of inflammatory conditions and diseases associated with CCR5 receptor.

The pharmacokinetic challenges associated with large molecules, proteins and peptides resulted in the establishment of programs to identify low molecular weight antagonists of CCR5. The efforts to identify chemokine modulators have been reviewed (W. Kazmierski et al. Biorg Med. Chem. 2003 11:2663-76; L. Agrawal and G. Alkhatib, Expert Opin. Ther. Targets 2001 5(3):303-326; Chemokine CCR5 antagonists incorporating 4-aminopiperidine scaffold, Expert Opin. Ther. Patents 2003 13(9):1469-1473; M. A. Cascieri and M. S. Springer, Curr. Opin. Chem. Biol. 2000 4:420-426, and references cited therein).

In U.S. Patent Publication 20050176703 published Aug. 11, 2005 S. D. Gabriel et al. disclosed 1-oxa-3,8-diaza-spiro[4.5]decan-2-one and 1-oxa-3,9-diaza-spiro[5.5]undecan-2-one derivatives which are CCR5 receptor antagonists.

### SUMMARY OF THE INVENTION

The present invention provides a compound according to formula I wherein

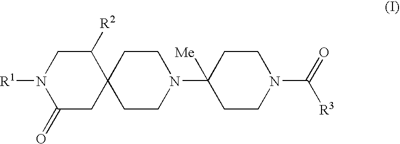

R¹ is tetrahydropyranyl-methyl, tetrahydrofuranyl-methyl, 4-C₁₋₆ alkoxy-cyclohexylmethyl, 4-hydroxy-cyclohexylmethyl, C₃₋₆ cycloalkyl-C₁₋₃ alkyl, or IIa to IId

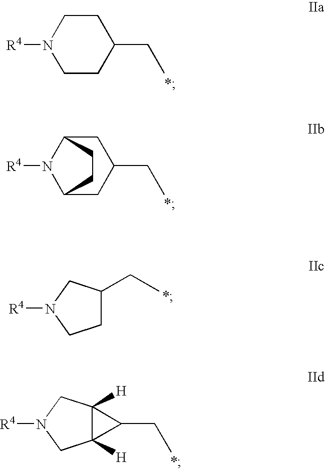

wherein:

R⁴ is —C(═O)OR⁵, —SO₂R⁵, C₁₋₆ acyl, C₁₋₆ haloalkyl;

said cycloalkyl is optionally independently substituted with one to three groups independently selected from the group consisting of hydroxy, C₁₋₆ alkoxy, C₁₋₃ alkyl, oxo, and halogen;

R² is C₁₋₆ alkyl, C₁₋₆ alkenyl, or C₁₋₄ alkoxy-C₁₋₃ alkyl;

R³ is selected from the group consisting of (a)-(e) and (f):
(a) 4,6-dimethyl-pyrimidin-5-yl;
(b) 4,6-dimethyl-2-trifluoromethyl-pyrimidin-5-yl;
(c) 2,4-dimethyl-pyridin-3-yl;
(d) 6-cyano-2,4-dimethyl-pridin-3-yl;
(e) 2,4-dimethyl-6-oxo-1,6-dihydro-pyridin-3-yl;
(f) 1,2,4-trimethyl-6-oxo-1,6-dihydro-pyridin-3-yl;

R⁵ is C₁₋₆ alkyl; or

mixtures of diastereomers, enantiomers or purified enantiomers or pharmaceutically acceptable salts thereof.

Compounds of formula I are CCR5 receptor antagonists which are useful for inhibiting HIV-1 viral entry and therefore for treating an HIV-1 infection. CCR5 antagonists according to formula I also are useful in modulating the immune response and therefore can be used to treat inflammatory disorders such as rheumatoid arthritis, asthma, COPD and transplant rejection exacerbated or caused by autoimmune responses.

The present invention also provides compositions comprising a compound of formula I admixed with at least one carrier, diluent or excipient which are useful for administering a compound of formula I to a patient afflicted with an HIV-1 infection or an inflammatory disorders exacerbated or caused by autoimmune activity.

### DETAILED DESCRIPTION OF THE INVENTION

The phrase “a” or “an” entity as used herein refers to one or more of that entity, for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R's can be carbon, both R's can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or [symbol] drawn through a bond each refer to the point of attachment of a functional group or other chemical moiety to the rest of the molecule of which it is a part. Thus, for example:

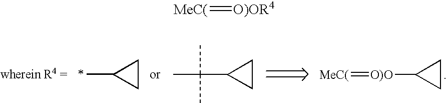

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R's can be carbon, both R's can be nitrogen, or one R″ can be carbon and the other nitrogen.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The term “alkyl” as used herein without further limitation denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

The term “alkylene” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkenyl” as used herein denotes an unsubstituted hydrocarbon chain radical having from 2 to 10 carbon atoms having one or two olefinic double bonds C₂₋₁₀ alkenyl” as used herein refers to an alkenyl composed of 2 to 10 carbons. Examples are vinyl, 1-propenyl, 2-propenyl(allyl) or 2-butenyl(crotyl).

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “alkoxyalkyl” as used herein refers to the radical R′R″—, wherein R′ is an alkoxy radical as defined herein, and R″ is an alkylene radical as defined herein with the understanding that the attachment point of the alkoxyalkyl moiety will be on the alkylene radical. C₁₋₆ alkoxyalkyl denotes a group wherein the alkyl portion is comprised of 1-6 carbon atoms exclusive of carbon atoms in the alkoxy portion of the group. C₁₋₃ alkoxy-C₁₋₆ alkyl denotes a group wherein the alkyl portion is comprised of 1-6 carbon atoms and the alkoxy group is 1-3 carbons. Examples are methoxymethyl, methoxyethyl, methoxypropyl, ethoxymethyl, ethoxyethyl, ethoxypropyl, propyloxypropyl, methoxybutyl, ethoxybutyl, propyloxybutyl, butyloxybutyl, t-butyloxybutyl, methoxypentyl, ethoxypentyl, propyloxypentyl including their isomers.

The term “haloalkyl” as used herein denotes a unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, difluoromethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl. The term “halogen” or “halo” as used herein means fluorine, chlorine, bromine, or iodine.

The term “acyl” or “alkylcarbonyl” as used herein denotes a group of formula —C(═O)R wherein R is hydrogen or lower alkyl as defined herein. The term C₁₋₆ acyl refers to a group —C(═O)R contain 1 to 6 carbon atoms. The C₁ acyl group is the formyl group wherein R═H and a C6 acyl group refers to hexanoyl when the alkyl chain is unbranched. The term “arylcarbonyl” as used herein means a group of formula C(═O)R wherein R is an aryl group; the term “benzoyl” as used herein an “arylcarbonyl” group wherein R is phenyl.

The term “oxo” as used herein refers to a double bonded oxygen (═O) linked to a carbonyl which together form a ketone or aldehyde. Thus cyclohexane with an oxo substituent is cyclohexanone.

The terms “oxetanyl”, “tetrahydrofuranyl” and “tetrahydropyranyl” refer to a four, five and six-membered non-fused heterocyclic ring respectively, each containing one oxygen atom. The term “pyridine” refers to a six-membered heteroaromatic ring with one nitrogen atom. The terms “pyrimidine”, “pyrazine” and “pyridazine” refer to a six-membered nonfused heteroaromatic ring with two nitrogen atoms disposed in a 1,3, a 1,4 and a 1,2 relationship respectively. The term “tetrahydro-pyranylmethyl” refers to a moiety of structure (i) where the methylene is attached to and carbon atom and “tetrahydro-furan-3-ylmethyl refers to a moiety of structure (ii).

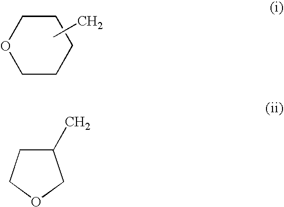

### Methods for Treating HIV-1 Infections

HIV-1 infects cells of the monocyte-macrophage lineage and helper T-cell lymphocytes by exploiting a high affinity interaction of the viral enveloped glycoprotein (Env) with the CD-4 antigen. The CD-4 antigen was found to be a necessary, but not sufficient requirement for cell entry and at least one other surface protein was required to infect the cells (E. A. Berger et al., Ann. Rev. Immunol. 1999 17:657-700). Two chemokine receptors, either the CCR5 or the CXCR4 receptor were subsequently identified as co-receptors along with CD4 which are required for infection of cells by the human immunodeficiency virus (HIV). The central role of CCR5 in the pathogenesis of HIV was inferred by epidemiological identification of powerful disease modifying effects of the naturally occurring null allele CCR5 Δ32. The Δ32 mutation has a 32-base pair deletion in the CCR5 gene resulting in a truncated protein designated Δ32. Relative to the general population, Δ32/Δ32 homozygotes are significantly more common in exposed/uninfected individuals suggesting the role of CCR5 in HIV cell entry (R. Liu et al., Cell 1996 86(3):367-377; M. Samson et al., Nature 1996 382(6593):722-725). The CD-4 binding site on the gp120 of HIV appears to interact with the CD4 molecule on the cell surface, and undergoes conformational changes which allow it to bind to another cell-surface receptor, such as CCR5 and/or CXCR-4. This brings the viral envelope closer to the cell surface and allows interaction between gp41 on the viral envelope and a fusion domain on the cell surface, fusion with the cell membrane, and entry of the viral core into the cell. Accordingly, an agent which could block chemokine receptors in humans who possess normal chemokine receptors should prevent infection in healthy individuals and slow or halt viral progression in infected patients.

RANTES and an analog chemically modified on the N-terminus, aminooxypentane RANTES, were found to block HIV entry into the cells. (G. Simmons et al., Science 1997 276:276-279). Other compounds have been demonstrated to inhibit the replication of HIV, including soluble CD4 protein and synthetic derivatives (Smith, et al., Science 1987 238:1704-1707), dextran sulfate, the dyes Direct Yellow 50, Evans Blue, and certain azo dyes (U.S. Pat. No. 5,468,469). Some of these antiviral agents have been shown to act by blocking the binding of gp120, the coat protein of HIV, to its target, the CD4 glycoprotein of the cell.

A-M. Vandamme et al. (Antiviral Chemistry & Chemotherapy, 1998 9:187-203) disclose current HAART clinical treatments of HIV-1 infections in man including at least triple drug combinations. Highly active anti-retroviral therapy (HAART) has traditionally consisted of combination therapy with nucleoside reverse transcriptase inhibitors (NRTI), non-nucleoside reverse transcriptase inhibitors (NNRTI) and protease inhibitors (PI). These compounds inhibit essential biochemical processes required for viral replication. While HAART has dramatically altered the prognosis for HIV infected persons, many drawbacks to the current therapy remain including highly complex dosing regimes and side effects which can be very severe (A. Carr and D. A. Cooper, Lancet 2000 356(9239):1423-1430). Moreover, these multidrug therapies do not eliminate HIV-1 and long-term treatment usually results in multidrug resistance, thus limiting their utility in long-term therapy. Development of new therapeutics which can be used in combination with NRTIs, NNRTIs, PIs and viral fusion inhibitors to provide better HIV-1 treatment remains a priority.

Typical NRTIs suitable for combination therapy include zidovudine (AZT; RETROVIR®); didanosine (ddl; VIDEX®); zalcitabine (ddC; HIVID®); stavudine (d4T; ZERIT®); lamivudine (3TC; EPIVIR®); abacavir (ZIAGEN®); adefovir dipivoxil [bis(POM)-PMEA; PREVON®]; lobucavir (BMS-180194), a nucleoside reverse transcriptase inhibitor disclosed in EP-0358154 and EP-0736533; BCH-10652, a reverse transcriptase inhibitor (in the form of a racemic mixture of BCH-10618 and BCH-10619) under development by Biochem Pharma; emitricitabine [(−)-FTC] in development by Triangle Pharmaceuticals; β-L-FD4 (also called β-L-D4C and named β-L-2′,3′-dicleoxy-5-fluoro-cytidene) licensed Vion Pharmaceuticals; DAPD, the purine nucleoside, (−)-β-D-2,6-diamino-purine dioxolane disclosed in EP-0656778 and licensed to Triangle Pharmaceuticals; and lodenosine (FddA), 9-(2,3-dideoxy-2-fluoro-β-D-threo-pentofuranosyl)adenine, an acid stable purine-based reverse transcriptase inhibitor under development by U.S. Bioscience Inc.

Approved NNRTIs include nevirapine (BI-RG-587; VIRAMUNE®); delaviradine (BHAP, U-90152; RESCRIPTOR®); efavirenz (DMP-266; SUSTIVA®) and etravirine (TMC-125, INTELENCE®). NNRTIs currently in clinical trials include TMC-278 (J. E. G. Guillemont et al., WO2003/016306), UK-453,061 (L. H. Jones et al., WO2002/085860), AR806 (J.-L. Girardet et al., WO2006/026356) and IDX899 (R. Storer et al. US2006074054). Still other NNRTIs include PNU-142721, a furopyridine-thio-pyrimidine under development by Pfizer; AG-1549 (formerly Shionogi #S-1153); 5-(3,5-dichlorophenyl)-thio-4-isopropyl-1-(4-pyridyl)methyl-1H-imidazol-2-ylmethyl carbonate disclosed in WO 96/10019; MKC-442 (1-(ethoxy-methyl)-5-(1-methylethyl)-6-(phenylmethyl)-(2,4(1H, 3H)-pyrimidinedione); and (+)-calanolide A (NSC-675451) and B, coumarin derivatives disclosed in U.S. Pat. No. 5,489,697.

Recently inhibitors of HIV-1 integrase have proved to been useful to treat HIV-1. N-substituted hydroxy pyrimidinone carboxamide inhibitors of HIV-1 integrase inhibitors have been disclosed by B. Crescenzi et al. in WO2003/035077, published May 1, 2003, and MK-0518 (raltegravir) has been approved by the FDA. GS 9137 (Elvitegravir) or JTK-303, licensed by Gilead Sciences from Japan Tobacco is undergoing Phase 2 trials. (A. Savarino, Expert Opin Investig Drugs. 2006 15(12):1507-22)

Typical suitable PIs include saquinavir (Ro 31-8959; INVIRASE®; FORTOVASE®); ritonavir (ABT-538; NORVIR®); indinavir (MK-639; CRIXIVAN®); nelfnavir (AG-1343; VIRACEPT®); amprenavir (141W94; AGENERASE®); lasinavir (BMS-234475); DMP-450, a cyclic urea under development by Triangle Pharmaceuticals; BMS-2322623, an azapeptide under development by Bristol-Myers Squibb as a 2nd-generation HIV-1 PI; ABT-378 under development by Abbott; and AG-1549 an imidazole carbamate under development by Agouron Pharmaceuticals, Inc.

Other antiviral agents include hydroxyurea, ribavirin, IL-2, IL-12, pentafuside. Hydroxyurea (Droxia), a ribonucleoside triphosphate reductase inhibitor shown to have a synergistic effect on the activity of didanosine and has been studied with stavudine. IL-2 (aldesleukin; PROLEUKIN®) is disclosed in Ajinomoto EP-0142268, Takeda EP-0176299, and Chiron U.S. Pat. No. RE 33,653, U.S. Pat. No. 4,530,787, 4,569,790, 4,604,377, 4,748,234, 4,752,585, and 4,949,314. Pentafuside (FUZEON®) a 36-amino acid synthetic peptide that inhibits fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred. Ribavirin, 1-β-D-ribofuranosyl-1H-1,2,4-triazole-3-carboxamide.

In addition to the potential for CCR5 modulators in the management of HIV infections, the CCR5 receptor is an important regulator of immune function and compounds of the present invention may prove valuable in the treatment of disorders of the immune system. Treatment of solid organ transplant rejection, graft v. host disease, arthritis, rheumatoid arthritis, inflammatory bowel disease, atopic dermatitis, psoriasis, asthma, allergies or multiple sclerosis by administering to a human in need of such treatment an effective amount of a CCR5 antagonist compound of the present invention is also possible.

### Methods for Treating Rheumatoid Arthritis

Modulators of the CCR5 receptor may be useful in the treatment of various inflammatory conditions. Rheumatoid arthritis is characterized by infiltration of memory T lymphocytes and monocytes into inflamed joints. As leukocyte chemotactic factors, chemokines play an indispensable role in the attraction of macrophages to various tissues of the body, a process which is essential for both inflammation and the body's response to infection. Because chemokines and their receptors regulate trafficking and activation of leukocytes which contribute to the pathophysiology of inflammatory and infectious diseases, agents which modulate CCR5 activity, preferably antagonizing interactions of chemokines and their receptors, are useful in the therapeutic treatment of such inflammatory diseases.

Elevated levels of CC chemokines, especially CCL2, CCL3 and CCL5, have been found in the joints of patients with rheumatoid arthritis and have been correlated with the recruitment on monocytes and T cells into synovial tissues (I. F. Charo and R. M. Ransohoff, New Eng. J. Med. 2006 354:610-621). T-cells recovered from synovial fluid of rheumatoid arthritis have been shown to express CCR5 and CXCR3. P. Gao et al., J. Leukocyte Biol. 2003 73:273-280) Met-RANTES is an amino-terminal modified RANTES derivative which blocks RANTES binding to the CCR1 and CCR5 receptors with nanomolar potency. (A. E. Proudfoot et al., J. Biol. Chem. 1996 271:2599-2603). The severity of arthritis in rat adjuvant-induced arthritis was reduced by the administration of Met-RANTES. In addition, the levels of pro-inflammatory cytokines TNF-α and IL-1, macrophage colony-stimulating factor, and RANKL were decreased in joints with adjuvant-induced arthritis in the Met-RANTES group compared with the control group. (S. Shahrara et al. Arthr. & Rheum. 2005 52:1907-1919) Met-RANTES has been shown to ameliorate the development of inflammation in an art recognized rodent model of inflammation, the collagen induced arthritis. (C. Plater-Zyberk et al. Immunol. Lett. 1997 57:117-120)

TAK-779 has also been shown to reduce both the incidence and severity of arthritis in the collagen-induced arthritis model. The antagonist inhibited the infiltration of inflammatory CCR5⁺ T-cells into the joint. (Y.-F. Yang et al., Eur. J. Immunol. 2002 32:2124-2132). Another CCR5 antagonist, SCH—X, was shown to reduce the incidence and severity of collagen-induced arthritis in rhesus monkeys. (M. P. M. Vierboom et al., Arthr. & Rheum. 2005 52(20):627-636).

In some inflammatory conditions compounds of the present invention may be administered in combination with other anti-inflammatory drugs which may have a alternative mode of action. Compounds which may be combined with CCR5 antagonists include, but are not limited to:

(a) a lipoxygenase antagonist or biosynthesis inhibitor such as an inhibitor of 5-lipoxygenase, leukotriene antagonists (e.g., zafirlukast, montelukast, pranlukast, iralukast, pobilukast, SKB-106,203), leukotriene biosynthesis inhibitors (e.g., zileuton, BAY-1005);

(b) a non-steroidal anti-inflammatory agent or cyclooxygenase (COX1 and/or COX2) inhibitor such as such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenarnic acid derivatives (flufenarnic acid, meclofenamic acid, mefenamic acid, niflumic acid and tolfenamic acid), biphenylearboxylic acid derivatives (diflunisal and flufenisal), oxicarns (isoxicarn, piroxicam, sudoxicam and tenoxican), salicylates (acetyl salicylic acid, sulfasalazine), pyrazolones (apazone, bezpiperylon, feprazone, mofebutazone, oxyphenbutazone, phenylbutazone) and celecoxib;

(c) a TNF inhibitor such as infliximab (REMICADE®), etanercept (ENBREL®), or adalimumab (HUMIRA®);

(d) anti-inflammatory steroids such as beclomethasone, methylprednisolone, betamethasone, prednisone, dexamethasone, and hydrocortisone; (e) immunomodulators such as cyclosporine, leflunomide (Arava®), azathioprine (Azasan®), penicillamine and levamisole;

(f) folate antagonists such as methotrexate;

(g) gold compounds such as aurothioglucose, gold sodium thiomalate or auranofin.

### Methods for Treating Transplant Rejection

Rejection following solid organ transplantation also is characterized by infiltration of T-cells and macrophages expressing the CCR5 receptor into the interstitial area. (J. Pattison et al., Lancet 1994 343:209-211) Renal transplant patients homozygous for the CCR5Δ32 deletion a significant survival advantage of patients heterozygous for the CCR5Δ32 deletion or homozygous wild type patients. (M. Fischerder et al., Lancet 2001 357:1758-1761) CCR5^{-/-} knockout mice showed significant prolong graft survival in after transplantation of heart and islet tissue. (W. Gao et al., Transplantation 2001 72:1199-1205; R. Abdi et al., Diabetes 2002 51:2489-2495. Blocking the CCR5 receptor activation has been found to significantly extend cardiac allograph survival. (W. W. Hancock et al., Curr. Opin. Immunol. 2003 15:479-486).

In treatment of transplant rejection or graft vs. host diseases CCR5 antagonists of the present invention may be administered in combination with other immunosuppressive agents including, but are not limited to, cyclosporine (SANDIMMUNE®), tacrolimus (PROGRAF®, FK-506), sirolimus (RAPAMUNE®, rapamycin), mycophenolate mofetil (CELLCEPT®), methotrexate, anti-IL-2 receptor (anti-CD25) antibodies such as daclizumab (ZENAPAX®) or basiliximab (SIMULECT®), anti-CD3 antibodies visilizumab (NUVION®) or muromonab (OKT3, ORTHOCLONE®).

### Methods for Treating Asthma and COPD

Antagonism of the CCR5 receptor has been suggested as a target to inhibit of progression of asthma and COPD by antagonism of Th1 activation: B. Ma et al., J. Immunol. 2006 176(8):4968-4978, B. Ma et al., J. Clin. Investig. 2005 115(12):3460-3472 and J. K. L. Walker etal., Am. J. Respir. Cell Mol. Biol. 2006 34:711-718.

In one embodiment of the present invention there is provided a compound according to formula I wherein R¹, R², R³, R⁴, and R⁵ are as described hereinabove. Substituent definitions in this and the following embodiments which are not specifically limited in the description of the embodiment retain the broadest scope defined in the Summary of the Invention. All the embodiments include pharmaceutically acceptable salts of the compounds of formula I.

In a second embodiment of the present invention there is provided a compound according to formula I wherein R¹ is cyclohexyl optionally substituted by C₁₋₆ alkoxy; R² is n-Bu and R³ is (a), (c) or (d).

In a third embodiment of the present invention there is provided a compound according to formula I wherein R¹ is tetrahydropyranyl-methyl or tetrahydrofuranyl-methyl; R² is n-Bu and R³ is (a), (c) or (d).

In a fourth embodiment of the present invention there is provided a compound according to formula I wherein R¹ is tetrahydropyranyl-methyl; R² is n-Bu and R³ is (a), (c) or (d).

In a fifth embodiment of the present invention there is provided a compound according to formula I wherein R¹ is tetrahydrofuranyl-methyl; R² is n-Bu and R³ is (a), (c) or (d).

In a sixth embodiment of the present invention there is provided a compound according to formula I wherein R¹ is IIa; R² is n-Bu and R³ is (a), (c) or (d) and R⁴ is C(═O)OR⁵, —SO₂R⁵ or C₁₋₆ acyl.

In a seventh embodiment of the present invention there is provided a compound selected from I-1 to I-15 in TABLE I.

In an eighth embodiment of the present invention there is provided a method for treating an human immunodeficiency virus (HIV-1) infection, or treating AIDS or ARC, in a patient in need thereof which comprises administering to the patient in need thereof a therapeutically effective amount of a compound according to formula I wherein R¹, R², R³, R⁴ and R⁵ are as defined herein above.

In a ninth embodiment of the present invention there is provided a method for treating an human immunodeficiency virus (HIV-1) infection, or treating AIDS or ARC, in a patient in need thereof which comprises co-administering to the patient in need thereof a therapeutically effective amount of a compound according to formula I wherein R¹, R², R³ R⁴, and R⁵ are as defined herein above together with one or more compound(s) selected from the group consisting of HIV-1 nucleoside reverse transcriptase inhibitors, non-nucleoside reverse transcriptase inhibitors, HIV-1 protease inhibitors, integrase inhibitors, and HIV-1 viral fusion inhibitors.

In a tenth embodiment of the present invention there is provided a method for treating rheumatoid arthritis, in a patient in need thereof which comprises administering to the patient a therapeutically effective amount of a compound according to formula I wherein R¹, R², R³ R⁴, and R⁵ are as defined herein above.

In an eleventh embodiment of the present invention there is provided a method for treating rheumatoid arthritis, in a patient in need thereof which comprises co-administering to the patient in need thereof a therapeutically effective amount of a compound according to formula I wherein R¹, R², R³, R⁴ and R⁵ are as defined herein above together with one or more anti-inflammatory or analgesic compounds.

In a twelfth embodiment of the present invention there is provided a method for treating asthma or COPD, in a patient in need thereof which comprises administering to the patient in need thereof a therapeutically effective amount of a compound according to formula I wherein R¹, R², R³, R⁴ and R⁵ are as defined herein above.

In a thirteenth embodiment of the present invention there is provided a method for treating solid organ transplant rejection, in a patient in need thereof which comprises administering to the patient in need thereof a therapeutically effective amount of a compound according to formula I wherein R¹, R², R³, R⁴ and R⁵ are as defined herein above.

In a fourteenth embodiment of the present invention there is provided a method for treating solid organ transplant rejection, in a patient in need thereof which comprises co-administering to the patient in need thereof a therapeutically effective amount of a compound according to formula I wherein R¹, R², R³, R⁴ and R⁵ are as defined herein above together with one or more anti-rejection drugs or immunomodulators.

In a fifteenth embodiment of the present invention there is provided a pharmaceutical composition comprising a compound according to formula I wherein R¹, R², R³, R⁴ and R⁵ are as defined herein above together with one or more carriers, excipients or diluents.

Commonly used abbreviations include: acetyl (Ac), azo-bis-isobutyrylnitrile (AIBN), atmospheres (Atm), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I

| | 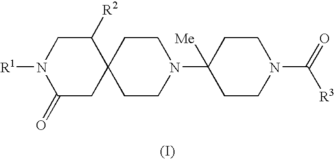 | | | | | IC₅₀ CCR5 |
|---|---|---|---|---|---|---|
| Cpd | | | | | | binding |
| No. | R¹ | R³ | R² | ms | mp | (μM) |
| I-1¹ | 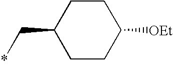 | 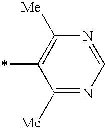 | n-Bu | 596 | | 0.0304 |
| I-2² | 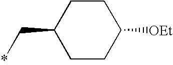 | 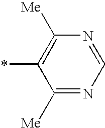 | n-Bu | 596 | | 0.0268 |
| I-3³ | 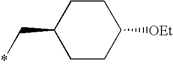 | 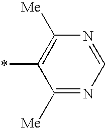 | n-Bu | | | 0.0329 |
| I-4⁴ | 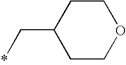 | 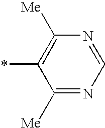 | n-Bu | 554 | 92.8- 100.1 | 0.0172 |
| I-5 | 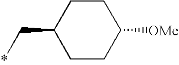 | 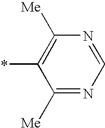 | n-Bu | 582 | | 0.0208 |
| I-6 | 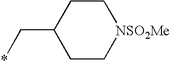 | 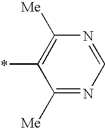 | n-Bu | 631 | | 0.0169 |
| I-7 | 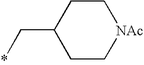 | 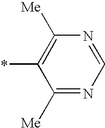 | n-Bu | 595 | | 0.0517 |
| I-8 | 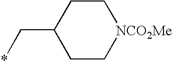 | 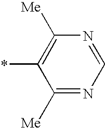 | n-Bu | 611 | | 0.0244 |
| I-9 | 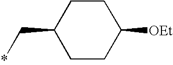 | 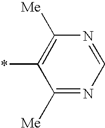 | n-Bu | 596 | | 0.032 |
| I-10 | 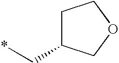 | 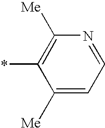 | n-Bu | 539 | | 0.0249 |
| I-11 | 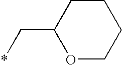 | 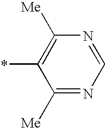 | n-Bu | 554 | | 0.0246 |
| I-12 | 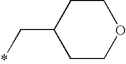 | 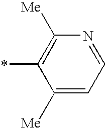 | n-Bu | 553 | | 0.0137 |
| I-13 | 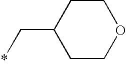 | 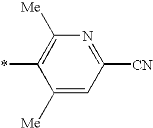 | n-Bu | 578 | | 0.0197 |
| I-14 | 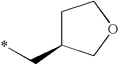 | 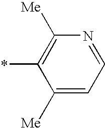 | n-Bu | 539 | | 0.0177 |
| I-15 | 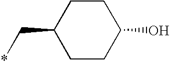 | 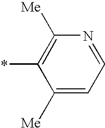 | n-Bu | 567 | | 0.0305 |

1. Isomeric mixture
2. Enantiomer 1
3. Enantiomer 2
4. The isomers could be separated on a (R,R)-Whelk-O chiral HPLC column eluting with MeOH at a flow rate of 1.4 mL/min.

Compounds of the present invention can prepared by from A-2 alkylation of the pyridone nitrogen and subsequently introducing and acylating the piperidine ring. A-2 was prepared from A-1a which, in turn, was prepared from (1-benzyl-piperidin-4-ylidene)-cyano-acetic acid ethyl ester by conjugate addition of ethyl caproate. Reduction of the ester with lithium pyrrolidinoborohydride (G. B. Fisher et al., Tetrahedron Lett. 1992 33(32):4533) afforded A-1b which was converted to the azide by treating A-1b with NaN₃ and DEAD. Treating the azide with Ph₃P resulted in an intramolecular Staudinger-aza Wittig reaction to afford A-2 after hydrolysis of the intermediate amidine.

Alcohols can be prepared by reduction of a carboxylic acid or carboxylic ester with a variety suitable reducing agent such as LiAlH₄, DIBAL-H, lithium amino borohydrides and BH₃ in an inert solvent, e.g. aliphatic hydrocarbons, such as hexane, heptane and petroleum ether; aromatic hydrocarbons, such as benzene, toluene, o-dichlorobenzene, and xylene; ethers, such as diethyl ether, diisopropyl ether, THF, diglyme and dioxane, preferably the ethers.

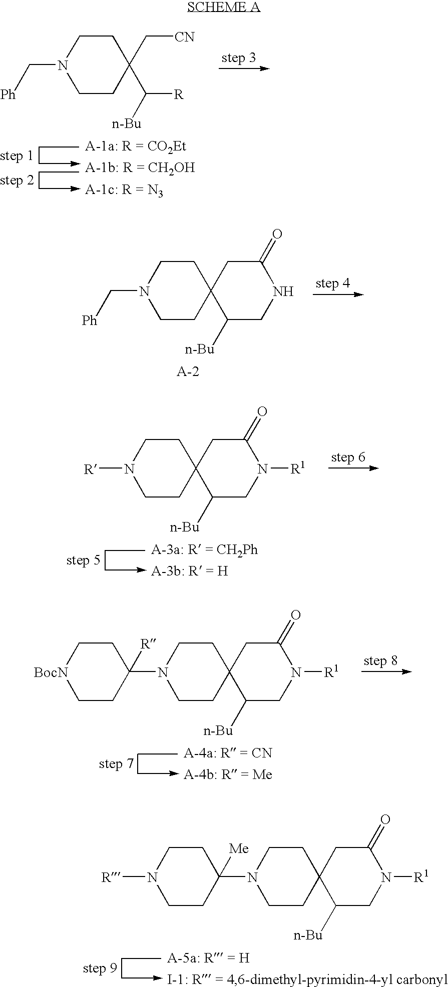

Methods for alkylation of amides under basic conditions (step 4) are well known to one skilled in the art. The reaction is typically carried out in aprotic solvents such as THF, DMF, DMSO, NMP and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

After cleavage of the benzyl protecting group from the N9 atom (step 5), the 4-methyl-N-Boc-piperidine moiety was introduced by Ti(O-i-Pr)₄ mediated condensation of the secondary amine A-3b with N-Boc-4-oxopiperidine (A-6) and trapping the intermediate imine with Et₂AlCN which results in the introduction of a nitrile at the 4-position (A-4a) which is, in turn, displaced with methyl magnesium bromide to afford A-4b. (A. Palani et al. J. Med. Chem. 2001 44(21):3339-42).

Removal of the Boc protecting group and acylation of the nitrogen affords the compounds of the present invention. Deprotection of the Boc group is carried out under acidic conditions such as TFA/DCM or HCl/dioxane.

Acylation of an amine can be effected by preparing an activated carboxylic acid such as an acid chloride or a symmetrical or mixed acid anhydride and reacting the activated derivative with the amines of formula A-5a in a solvent such as DMF, DCM, THF, with or without water as a co-solvent, and the like at temperatures between 0° and 60° C. generally in the presence of a base such as Na₂CO₃, NaHCO₃, K₂CO₃, DIPEA, TEA or pyridine. Carboxylic acids are converted into their acid chlorides using standard reagents well known to one skilled in the art, such as thionyl chloride, oxalyl chloride, phosphoryl chloride and the like. Those reagents can be used in presence of bases such as DIPEA, TEA or pyridine in inert solvent such as DCM or DMF.

Alternatively a carboxylic acid can be converted in situ into activated acid derivative by procedures developed for peptide synthesis which are well known to those skilled in the art. These activated acids were reacted directly with the amines of formula A-5a to give the compounds of formula I. Common coupling protocols employ an activating agent like EDCI or DCC, HOBt, benzotriazol-1-yloxy-tris-(dimethylamino)phosphonium hexafluorophosphate (BOP), bromo-tris-pyrrolidinophosphonium hexafluorophosphate (PyBrOP), or 2-fluoro-1-methylpyridinium p-toluenesulphonate (Mukaiyama's reagent) and the like with or without a base such NMM, TEA or DIPEA in an inert solvent such as DMF or DCM at temperatures between 0° C. and 60° C. The reaction may alternatively be carried out in presence of O-(7-azabenzotriazol-1-yl)-N,N,N′,N′-tetramethyluronium hexafluorophosphate (HATU) or 1-hydroxy-7-azabenzotriazole (HOAt) and TEA or DIPEA in DMF, DCM or THF. Acylation of amines (J. March, supra pp. 417-425; H. G. Benz, Synthesis of Amides and Related Compounds in Comprehensive Organic Synthesis, E. Winterfeldt, ed., vol. 6, Pergamon Press, Oxford 1991 pp. 381-411) has been reviewed.

The sequence of the reaction steps is not critical and introduction of N9 amide can be carried out prior to the alkylation of the amide as depicted in SCHEME B utilizing analogous reaction conditions.

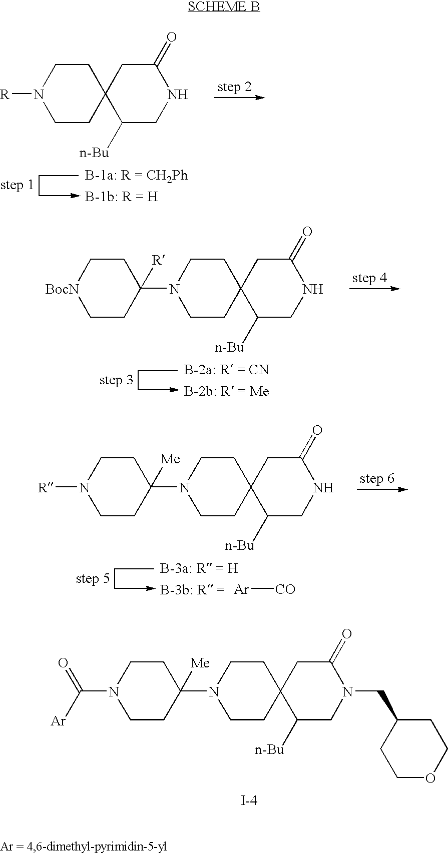

Compounds of the present invention with a pendant functionalized piperidine substituent were prepared (SCHEME C) by alkylation of B-3b with 4-bromomethyl piperidine 1-carboxylic acid tert-butyl ester. Deprotection of the amine and acylation or sulfonylation of the resulting secondary amine affords compounds of the present invention. The cyclohexanol derivative I-15 was prepared by alkylation of B-3b with toluene-4-sulfonic acid 4-(tert-butyl-dimethyl-silanyloxy)-cyclohexylmethyl ester which was deprotected in the final step.

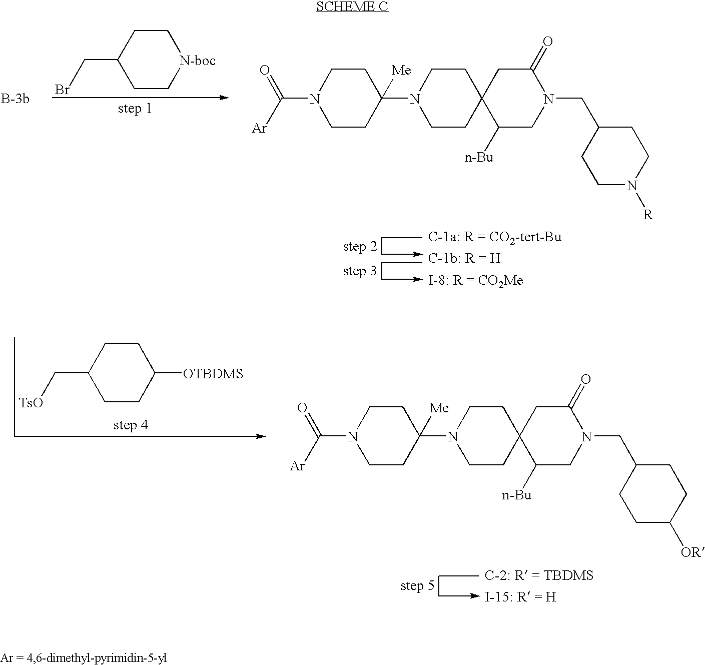

### Dosage and Administration

The compounds of the present invention may be formulated in a wide variety of oral administration dosage forms and carriers. Oral administration can be in the form of tablets, coated tablets, dragees, hard and soft gelatine capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w).

The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone(1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and expcipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### REFERENTIAL EXAMPLE

### 2-(1-Benzyl-4-cyanomethyl-piperidin-4-yl)-hexanoic acid ethyl ester

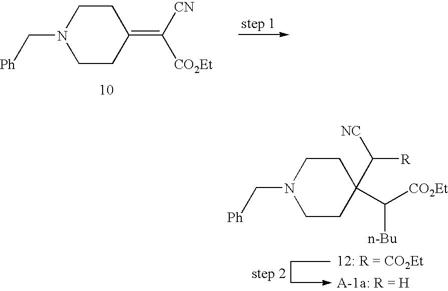

step 1—To a solution of diisopropylamine (4.88 mL, 34.9 mmol) in THF (100 mL) cooled to −78° C. was added n-BuLi (2.5 M in hexane, 13.3 mL, 33.3 mmol) and the reaction was stirred for 15 min. The dry-ice acetone bath was removed and stirring was continued for another 20 min then the reaction mixture was re-cooled to −78° C. To the LDA solution was added dropwise via a syringe over 10 min a solution of ethyl carproate (5.5 mL, 33.3 mmol) in THF (30 mL) which was pre-cooled to −78° C. The reaction was stirred at −78° C. for 40 min. A solution of 10 (8.6 g, 30 mmol; CASRN 1463-52-1) in THF (30 mL) was added via a syringe. The reaction mixture was poured into a mixture of H₂O and EtOAc. The organic layer was separated and the aqueous layer was extracted with EtOAc. The combined organic extracts were dried (MgSO₄), filtered, and concentrated. The residue was purified by SiO₂ column chromatography eluting with an EtOAc/hexane gradient (20% to 40% EtOAc over 30 min) to afford 11.71 g (90%) of a diastereomeric mixture of 12 as an oil: ¹HNMR (CDCl₃, 300 MHz) δ 7.32-7.24 (m), 4.30-4.08 (m), 3.51 (s), 2.81-2.26 (m), 2.15-1.95 (m), 1.90-1.65 (m), 1.40-1.15 (m), 0.91-0.85 (m); IR (neat film) 3062, 3027, 2958, 2873, 2810, 2769, 2246, 1736, 1604, 1495, 1454, 1370, 1320, 1249, 1181, 1074, 1030, 857, 740, 699 cm⁻¹; MS calc'd for C₁₅H₃₇N₂O₄ [M+H]⁺429. Found, 429.

step 2—A suspension of 12 (15.46 g, 36.1 mmol) and LiCl (3.06 g, 72.2 mmol) in DMSO (100 mL) and H₂O (10 mL) was heated at 200° C. for 1.5 h. After cooling to RT, the content was diluted with EtOAc and the solution washed with 50% aqueous saturated brine. The organic layer was separated. The aqueous layer was twice extracted with EtOAc. The combined extracts were dried (MgSO₄), filtered, and concentrated. The residue was purified by SiO₂ chromatography eluting with an EtOAc/hexane gradient (10% to 50% EtOAc over 36 min) to give 11.5 g of A-1a as an oil: Calc'd for C₁₉H₂₅N₂O₂: C, 74.12%; H, 9.05; N, 7.86. Found: C, 73.71; H, 8.89; N, 7.74.

### Example 1

### 5-Butyl-9-[1-(4,6-dimethyl-pyrimidine-5-carbonyl)-4-methyl-piperidin-4-yl]-3-(4-ethoxy-cyclohexylmethyl)-3,9-diaza-spiro[5.5]undecan-2-one (I-1; SCHEME A)

step 1—To a solution of compound A-1a (8.08 g, 22.7 mmol) in THF (100 mL) at RT was added lithium pyrrolidinoborohydride (1 M in THF, 87 mL, 87 mmol). The reaction was stirred at RT overnight, cooled to 0° C., and quenched with 1N aqueous NaOH. The resulting mixture was diluted with a solution of brine/28% aqueous NH₄OH (5:1) and thrice extracted with EtOAc. The combined organic layers were dried (MgSO₄), filtered and concentrated. The residue was purified by SiO₂ flash chromatography eluting with a gradient of DCM and a DCM/MeOH/28% aqueous NH₄OH solution (60:10:1; 90% to 50% DCM over 30 min) to afford 6.12 g (86%) of A-1b as a pale yellow oil: ¹HNMR (CDCl₃, 300 MHz) δ 7.32-7.22 (m, 5H), 3.79 (d, J=3.7 Hz, 2H), 3.50 (s, 2H), 2.65-2.58 (m, 4H), 2.26-2.16 (m, 2H), 1.86-1.77 (m, 1H), 1.74-1.61 (m, 4H), 1.51-1.20 (m, 6H), 0.91 (t, J=7.0 Hz, 3H); ¹³C NMR (CDCl₃, 300 Hz) δ 138.1, 129.1, 128.2, 127.1, 119.2, 63.2, 61.1, 49.0, 46.8, 36.3, 33.0, 31.0, 25.3, 22.9, 22.7, 14.1; IR (neat film) 3488, 3062, 3028, 2929, 2871, 2811, 2769, 2241, 1494, 1454, 1397, 1368, 1344, 1316, 1253, 1122, 1075, 1029, 961, 793, 744, 699 cm⁻¹; HRMS Calc'd for C₂₀H₃₁N₂O [M+H]⁺ 315.2436. Found: 315.2393.

step 2—To a solution of A-1b (17.5 g, 55.7 mmol) and PPh₃ (16.1 g, 61.3 mmol) in THF (70 mL) at RT was added DEAD (neat, 10.6 mL, 67.4 mmol). After stirring at RT for 10 min, diphenyl phosphoryl azide (neat, 14.6 mL, 67.4 mmol) was added. The mixture was stirred overnight and concentrated under reduced pressure. The residue was purified by SiO₂ flash chromatography eluting with a EtOAc/hexane gradient (15% to 50% over 40 min) to afford 10.25 g (54%) of A-1c as a clear viscous oil: ¹HNMR (CDCl₃, 300 MHz) δ 7.33-7.20 (m, 5H), 5.12-4.97 (m, 1H), 3.58-3.42 (m, 4H), 2.65-2.58 (m, 2H), 2.59 (d, J=4.4 Hz, 2H), 2.25-2.15 (m, 2H), 1.8-1.2 (m, 11H), 0.92 (t, J=7.0 Hz, 3H); ¹³C NMR (CDCl₃, 300 Hz) δ 138.1, 129.0, 128.3, 127.1, 118.4, 63.1, 50.7, 48.9, 44.9, 36.4, 32.8, 32.6, 30.7, 26.6, 22.8, 22.1, 21.9, 21.7, 14.0; IR (neat film) 3432, 3028, 2955, 2872, 2809, 2768, 2098, 1740, 1493, 1455, 1374, 1264, 1183, 1103, 1028, 965, 741 cm⁻¹; HRMS Calc'd for C₂₀H₃₀N₅ [M+H]⁺ 340.2501. Found: 340.2516.

step 3—To a solution of A-1c (2.04 g, 6 mmol) in THF (40 mL) at RT was added PPh₃ (2.36 g, 9 mmol). The reaction mixture was stirred at 70° C. for 1 h and concentrated under reduced pressure. The residue was taken up in concentrated aqueous HCl (ca. 40 mL) and heated at 100° C. in a sealed pressure tube over three nights. After cooling to RT, the content was poured into a beaker and quenched with solid Na₂CO₃. The mixture was further diluted with water and thrice extracted with EtOAc. The combined organic layers were dried (Na₂SO₄), filtered and concentrated. The residue was purified by SiO₂ chromatography eluting with a gradient of DCM and a DCM/MeOH/28% aqueous NH₄OH solution (60:10:1; 80% to 30% DCM over 40 min) to afford 1.57 g (82% over two steps) of A-2 as a white solid: ¹HNMR (CDCl₃, 300 MHz) δ 7.30-7.20 (m, 5H), 6.85 (s, 1H), 3.51 (s, 2H), 3.41 (dd, J=1.5, 4.7 Hz, 1H), 3.10-3.02 (m, 1H), 2.70-2.60 (m, 2H), 2.41-2.13 (m, 4H), 1.80-1.13 (m, 11H), 0.89 (t, J=7.1 Hz, 3H); ¹³C NMR (CDCl₃, 300 Hz) δ 172.4, 138.6, 129.9, 129.6, 128.6, 128.3, 127.7, 127.4, 63.7, 49.3, 49.2, 42.5, 41.5, 38.6, 35.2, 33.8, 31.8, 30.9, 26.1, 23.2, 14.4; IR (neat film) 3421, 3195, 3060, 2948, 2932, 2869, 2799, 2762, 1670, 1505, 1451, 1411, 1366, 1341, 1313, 1121, 736 cm⁻¹; HRMS Calc'd for C₂₀H₃₁N₂O [M+H]⁺ 315.2436. Found: 315.2433.

step 4—To a suspension of A-2 (0.243 g, 0.77 mmol), NaOH (pearl, 0.154 g, 3.85 mmol), K₂CO₃ (0.117 g, 0.85 mmol) and tetrabutylammonium bromide (0.026 g, 0.08 mmol) in toluene (2 mL) was added trans-4-ethoxy-cyclohexylmethyl toluene-4-sulfonate. The reaction mixture was heated at 90° C. for 3 d. After cooling to RT, the content was diluted with brine/water (1:1) and thrice extracted with EtOAc. The combined organic layers were dried (MgSO₄), filtered and concentrated. The residue was purified by SiO₂ chromatography eluting with a gradient of DCM and a DCM/MeOH/28% aqueous NH₄OH solution (60:10:1; 80% to 40% DCM over 30 min) to afford 0.46 g of A-3a (R=trans-ethoxy-cyclohexyl-4-methyl) as a impure clear oil, which was used in the next step without further purification. MS Calc'd for C₂₉H₄₇N₂₂ [M+H]⁺ 455. Found: 455.

step 5—A suspension of A-3a from step 4 (0.46 g) and Pd(OH)₂/C (20 wt %, 500 mg) in MeOH (15 mL) was shaken under a hydrogen atmosphere (60 psi) in a Parr apparatus at RT for 3 h. The content was filtered through SOLKA FLOC® and concentrated to afford 0.316 g of crude A-3b as a foam: MS Calc'd for C₂₂H₄₁N₂O₂ [M+H]⁺ 365. Found: 365.

steps 6 & 7—To a solution of A-3b (0.316 g, assuming 100% purity, 0.87 mmol) and N-Boc-4-piperidone (0.19 g, 0.96 mmol) in DCM (5 mL) under argon was added Ti(O-i-Pr)₄ (0.3 mL, 1.04 mmol). The reaction was stirred at RT overnight then Et₂AlCN (1.0 M in toluene, 2.2 mL, 2.2 mmol) was added dropwise. After stirring at RT for 5 h, the reaction mixture was cooled to 0° C. and poured into a mixture of EtOAc (10 mL) and saturated aqueous NaHCO₃ (2 mL) at 0° C. The mixture was vigorously stirred at RT for 1 h, filtered through a plug of CELITE®. The filtrate was washed with brine, dried (MgSO₄), filtered and concentrated to afford 0.53 g of crude A-4a as a clear oil.

The oil was dissolved in anhydrous THF (10 mL) and cooled to 0° C. A solution of MeMgBr (3 M in Et₂O, 1.54 mL, 4.63 mmol) was added dropwise. The bath was removed and the reaction was stirred at RT overnight. The content was cooled with an ice bath and quenched with saturated aqueous NH₄Cl. The mixture was made basic with 28% aqueous NH₄OH and extracted with EtOAc. The combined organic layer was dried (MgSO₄), filtered and concentrated. The residue was purified by SiO₂ chromatography eluting with a gradient of DCM and a DCM/MeOH/28% aqueous NH₄OH solution (60:10:1; 80% to 40% DCM over 30 min) to afford 0.13 g of A-4b as a clear oil: MS calc'd for C₃₃H₆₀N₃O₄ [M+H]⁺ 562; Found: 562.

step 8—To a solution of A-4b (0.13 g, 0.23 mmol) in DCM (3.2 mL) at RT was added TFA (0.8 mL). The reaction was stirred at RT for 1 h, quenched with ice-cold saturated aqueous NaHCO₃, and extracted with EtOAc. The aqueous layer was concentrated with DCM with a continuous extractor overnight. The combined organic layers were dried (MgSO₄), filtered and concentrated to afford 98 mg of crude A-5a, which was used in the next step without further purification: MS calc'd for C₂₈H₅₂N₃O₂ [M+H]⁺ 462; Found: 462.

step 9—To a mixture of A-5a (98 mg, assuming 100% purity, 0.21 mmol), 4,6-dimethyl-pyrimidine-5-carboxylic acid (48 mg, 0.32 mmol), EDCI (92 mg, 0.42 mmol), HOBt hydrate (65 mg, 0.48 mmol) at RT were added sequentially DCM (6 mL) and DIPEA (0.67 mL, 3.8 mmol). The mixture was stirred at RT overnight, quenched with saturated aqueous NaHCO₃, and extracted with EtOAc. The combined extracts were dried (Na₂SO₄), filtered, and concentrated in vacuo. The residue was purified on a preparative SiO₂ chromatography plate and developed with a solution of 60% DCM and 40% DCM/MeOH/28% aqueous NH₄OH (60:10:1) to afford 40 mg of A-1 as a white powder: MS calc'd for C₃₅H₅₈N₅O₃ [M+H]⁺ 596; Found, 596.

The optical isomers I-2 and I-3 were separated by (R,R)-Whelk-O chiral HPLC column (Regis Technologies, Inc.) eluting with MeOH at a flow rate of 1.2 mL/min. The two isomers had retention times of 9.3 min and 10.9 min.

Compound 1-12 can be prepared analogously except in step 4, 4-bromomethyl tetrahydropyran was used in place of trans-4-ethoxy-cyclohexylmethyl toluene-4-sulfonate and in step 9, 6-cyano-2,4-dimethyl-nicotinic acid was used in place of 4,6-dimethyl pyrimidine 5-carboxylic acid. MS calc'd for C₃₄H₅₂N₅O₃ [M+H]⁺ 578; Found, 578.

### Example 2

### 5-Butyl-9-[1-(4,6-dimethyl-pyrimidine-5-carbonyl)-4-methyl-piperidin-4-yl]-3-(4-ethoxy-cyclohexylmethyl)-3,9-diaza-spiro[5.5]undecan-2-one (I-4, SCHEME B)

step 1—A suspension of B-1a (0.545 g) and Pd(OH)₂/C (20 wt %, 0.7 g) in EtOH (20 mL) was shaken in a hydrogen atmosphere (60 psi) in a Parr apparatus at RT for 4.5 h. The content was filtered through CELITE® and concentrated to afford 0.465 g of crude B-1b as a light brown solid: MS Calc'd for C₁₃H₂₅N₂O [M+H]⁺ 225. Found: 225.

step 2 & 3—To a solution of B-1b (0.447 g, assuming 100% purity, 2 mmol) and N-Boc-4-piperidone (0.438 g, 2.2 mmol) in DCM under argon was added Ti(O-i-Pr)₄ (0.94 mL, 3.2 mmol). The reaction was stirred at RT overnight then Et₂AlCN (1.0 M in toluene, 5 mL, 5 mmol) was added dropwise. After stirring at RT for 4.5 h, the reaction mixture was cooled to 0° C. and poured into a mixture of EtOAc (20 mL) and saturated aqueous NaHCO₃ (4 mL) at 0° C. The mixture was vigorously stirred at RT for 1 h, filtered through a plug of CELITE. The filtrate was washed with brine, dried (MgSO₄), filtered and concentrated to afford 0.9 g of crude B-2a as a foam.

The foam was dissolved in anhydrous THF (20 mL) and cooled to 0° C. A solution of MeMgBr (3 M in Et₂O, 3.3 mL, 9.9 mmol) was added dropwise. The bath was removed and the reaction was stirred at RT overnight. The reaction mixture was cooled with an ice bath and quenched with saturated aqueous NH₄Cl. The mixture was made basic with saturated aqueous NaHCO₃ and extracted with EtOAc. The combined organic layer was dried (MgSO₄), filtered and concentrated. The residue was purified by SiO₂ chromatography eluting with a gradient of DCM and DCM/MeOH/28% aqueous NH₄OH (60:10:1; 70% to 30% DCM over 20 min) to afford 0.822 g of B-2b as a white solid: MS calc'd for C₂₄H₄₄N₃O₃ [M+H]⁺ 422; Found: 422.

step 4—To a flask containing B-2b (0.69 g, assuming 100% purity, 1.64 mmol) was added a solution of HCl in dioxane (4M, 5 mL, 20 mmol) followed by dioxane (5 mL). The heterogeneous reaction mixture was stirred at RT for 1 h and concentrated under reduced pressure to afford crude amine B-3a, which was used in the next step without further purification. MS calc'd for C₁₉H₂₆N₃O [M+H]⁺ 322; Found: 322.

step 5—To a mixture of B-3a from step 4, 4,6-dimethyl-pyrimidine-5-carboxylic acid (374 mg, 2.46 mmol), EDCI (630 mg, 3.28 mmol), HOBt hydrate (443 mg, 3.28 mmol) at RT were added sequentially DCM (20 mL) and DIPEA (2.8 mL, 16 mmol). The mixture was stirred at RT overnight, quenched with saturated aqueous NaHCO₃, and extracted with EtOAc. The combined extracts were dried (Na₂SO₄), filtered, and concentrated in vacuo. The residue was purified by SiO₂ chromatography eluting with a gradient of DCM and DCM/MeOH/28% aqueous NH₄OH (60:10:1; 70% to 20% DCM over 25 min) to afford 403 mg of B-3b as an off-white foam (54% yield over two steps): MS Calc'd for C₂₆H₄₂N₅O₂ [M+H]⁺ 456; Found, 456.

step 6—A mixture of B-3b (0.4 g, 0.88 mmol), NaOH (pearl, 0.21 g, 5.28 mmol), K₂CO₃ (0.134 g, 0.97 mmol), tetrabutylammonium bromide (0.043 g, 0.13 mmol) and 4-bromomethyl tetrahydropyran (0.47 g, 2.64 mmol) in toluene (4 mL) was heated at 90° C. for 18 h. After cooling to RT, the content was diluted with brine/water (1:1) and thrice extracted with EtOAc. The combined organic extracts were dried (MgSO₄), filtered and concentrated. The residue was purified by SiO₂ chromatography eluting with a gradient of DCM and DCM/MeOH/28% aqueous NH₄OH (60:10:1; 80% to 30% DCM over 30 min, then 30% DCM for 5 min) to afford 0.18 g of I-4 as a white foam: MS Calc'd for C₃₂H₅₂N₅O₃ [M+H]⁺ 554; Found: 554.

I-5 can be prepared analogously except in step 6, 4-bromomethyl tetrahydropyran was replaced with trans-4-methoxy-cyclohexylmethyl p-toluenesulfonate: MS calc'd for C₃₄H₅₅N₅O₃ [M+H]⁺ 582; Found, 582.

I-9 can be prepared analogously except in step 6, 4-bromomethyl tetrahydropyran was replaced with cis-4-methoxy-cyclohexylmethyl p-toluenesulfonate: MS calc'd for C₃₅H₅₈N₅O₃ [M+H]⁺ 596; Found, 596.

I-11 can be prepared analogously except in step 6, 4-bromomethyl tetrahydropyran was replaced with racemic 2-bromomethyl tetrahydropyran (CASRN 34723-82-5): MS calc'd for C₃₂H₅₂N₅O₃ [M+H]⁺ 554; Found, 554.

I-10 can be prepared analogously except in step 5, 4,6-dimethyl pyrimidine 5-carboxylic acid was replaced with 2,4-dimethyl-nicotinic acid and in step 6, 4-bromomethyl tetrahydropyran was replaced with (R)-tetrahydrofuran-3-ylmethyl p-toluenesulfonate (CASRN 726180-98-9): MS calc'd for C₃₂H₅₁N₄O₃ [M+H]⁺ 539; Found, 539.

I-12can be prepared analogously except in step 5, 4,6-dimethyl pyrimidine 5-carboxylic acid was replaced with 2,4-dimethyl-nicotinic acid: MS calc'd for C₃₃H₅₃N₄O₃ [M+H]⁺ 553;. Found, 553.

### Example 3

### 4-{1-Butyl-9-[1-(4,6-dimethyl-pyrimidine-5-carbonyl)-4-methyl-piperidin-4-yl]-4-oxo-3,9-diaza-spiro[5.5]undec-3-ylmethyl}-piperidine-1-carboxylic acid methyl ester (I-8, SCHEME C)

step 1—The intermediate C-1a can be prepared from B-3b in accord with the procedure in step 6 of experiment 3 except replacing 4-bromomethyl tetrahydropyran was replaced with 4-bromomethyl piperidine 1-carboxylic acid tert-butyl ester. MS calc'd for C₃₇H₆₁N₆O₄ [M+H]⁺ 653; Found, 653.

step 2—Deprotection of the Boc protecting group of C-1a can be carried out in accord with the procedure in step 4 of experiment 3 to afford C-1b: MS calc'd for C₃₂H₅₃N₆O₂ [M+H]⁺ 553; Found, 553.

step 3—To a solution of C-1b (68 mg, 0.12 mmol) and TEA (0.051 mL, 0.37 mmol) in DCM (2 mL) at 0° C. was added methyl chloroformate (0.0116 mL, 0.15 mmol). The reaction mixture was stirred at RT for 1 h, poured into saturated aqueous NaHCO₃ and thrice extracted with DCM. The combined organic layers were dried (MgSO₄), filtered and concentrated. The residue was purified on a preparative TLC plate developed with a solution of DCM and DCM:MeOH:28% aqueous NH₄OH (60:10:1) (45% DCM) to afford 36 mg of I-8 as an oil: MS calc'd for C₃₄H₅₅N₆O₄ [M+H]⁺ 611; Found, 611.

Compound I-6 can be prepared analogously except in step 3, methyl sulfonyl chloride was used in place of methyl chloroformate: MS calc'd for C₃₃H₅₅N₆O₄S [M+H]⁺ 631; Found, 631.

Compound I-7 can be prepared analogously except in step 3, acetyl chloride was used in place of methyl chloroformate: MS calc'd for C₃₀H₄₇N₆O₃ [M+H]⁺ 539; Found, 539.

### Example 4

### 5-Butyl-9-[1-(2,4-dimethyl-pyridine-3-carbonyl)-4-methyl-piperidin-4-yl]-3-(4-hydroxy-cyclohexylmethyl)-3,9-diaza-spiro[5.5]undecan-2-one (I-15)

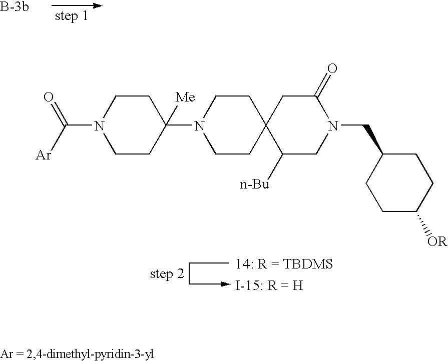

Compound 14 can prepared following procedure in experiment 2 except in step 5, 4,6-dimethyl pyrimidine 5-carboxylic acid is replaced with 2,4-dimethyl-nicotinic acid and in step 6, 4-bromomethyl tetrahydropyran is replaced with trans-4-tert-butyldimethylsilanyloxy cyclohexylmethylp-toluenesulfonate: R_{f}=0.39 (50% DCM/DCM:MeOH:28% aqueous NH₄OH (60:10:1)); ¹H NMR (400 MHz): 8.31 (d, J=1.36Hz, 1H), 6.94 (t, J=4.76Hz, 1H), 4.10 (m, 1H), 3.48 (m, 2H), 3.25 (m, 3H), 3.10 (m, 1H), 2.97 (m, 2H), 2.67 (m, 1H), 2.54 (m, 1H), 2.44 (d, J=11.1 Hz, 3H), 2.35 (m, 2H), 2.23 (d, J=9.6 Hz, 3H), 1.93 (m, 1H), 1.82 (m, 2H), 1.72 (m, 1H), 1.60 (m, 4H), 1.1-1.5 (m, 17H), 0.88 (m, 7H), 0.83 (s, 9H), 0.01 ppm (s, 6H).

step 2—To neat silyl ether 14 (0.071 mmol, 0.0486 g) was added 75% aqueous acetic acid (10 mL) and the resulting reaction mixture was stirred at RT overnight (16 h). The HOAc was neutralized with saturated aqueous NaHCO₃ and extracted with DCM. The combined organic layers were washed with.brine, dried (MgSO₄), filtered and concentrated in vacuo. The crude product was purified on a preparative TLC plate developed with a solution containing DCM and a solution of DCM:MeOH:28% aqueous NH₄OH (60:10:1) (40% DCM) to afford 0.0226 g (57%) of I-15 as a white foam: MS calc'd for C₃₄H₅₅N₄O₃ [M+H]⁺ 567; Found, 567.

### Example 5

### 5-Butyl-9-[1-(2,4-dimethyl-pyridine-3-carbonyl)-4-methyl-piperidin-4-yl]-3-[(R)-1-(tetrahydro-furan-3-yl)methyl]-3,9-diaza-spiro[5.5]undecan-2-one (I-14)

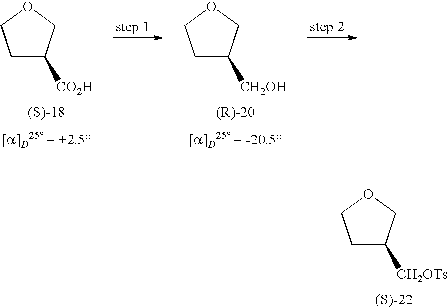

step 1—(S)-Tetrahydro-3-furanoic acid (3.3 g, 29.1 mmol) was dissolved in freshly distilled THF (15 mL) and added to a slurry of NaBH₄ (2.6 g, 69 mmol) in freshly distilled THF (15 mL) cooled to 0° C. and maintained under a N₂ atmosphere. The mixture was stirred for 10 min and then a solution of I₂ (7.3 g, 29 mmol) in anhydrous THF (15 mL) was added dropwise over a 30 min period. When gas evolution ceased the solution was heated at reflux for 12 h. The reaction mixture was cooled, the solvent evaporated and the residue was taken up in 20% aqueous KOH and stirred for 4 h at RT. The aqueous solution was continuously extracted with DCM for 2 d and the resulting extract was dried (MgSO₄), filtered and evaporated to afford 2.5 g of (R)-20: MS=(M+H)=103; NMR=¹H nmr δ 3.93-3.51 (m, 6H), 2.2-2.0 (m, 1H), 1.98-1.71 (m, 2H).

step 2—To a solution of (R)-20 (2.5 g, 24.4 mmol), TEA (50 mL), DMAP (149 mg) and DCM (50 mL) was added portionwisep-toluenesulfonyl chloride (5.1 g, 26.9 mmol). The reaction mixture was stirred overnight at RT under a N₂ atmosphere. The solvent was removed, the residue was dissolved in EtOAc and washed with water. The organic layer was dried (MgSO₄), filtered and evaporated. The crude material was purified by SiO₂ chromatography eluting with 30% EtOAc/hexane to afford 4.9 g of (S)-22: (M+H)=257.

The synthesis of I-14 can be carried out in analogy to the procedure described in example 2, except in step 6, 4-bromomethyl tetrahydropyran was replaced with (S)-22: MS calc'd for C₃₂H₅₁N₄O₃ [M+H]⁺ 539; Found, 539.

### Example 6

### Human CCR5 Receptor-Ligand Binding Assay Protocol

Human CCR5 receptor (Genebank ID: 29169292) was cloned into mammalian expression vector, pTarget (Promega). The construct was transfected into CHO-G_{α16} cells by using Fugene Reagent (Roche). Clones were selected under antibiotic pressure (G418 and Hygromycin) and sorted 4 times with a fluorescence activates cell sorter and a monoclonal antibody specific for CCR5 receptor (BD Biosciences Pharmigen, Mab 2D7, Cat. No. 555993). The clone with highest expression (100,000 copies per cell) was chosen for the binding assays.

Adherent cells in 225 mL tissue culture flask (˜90% confluent) were harvested using 1 mM EDTA in PBS (phosphate-buffered saline) without Ca²⁺ and Mg²⁺. Cells were washed twice with PBS containing no Ca²⁺ and Mg²⁺. CHO-G_{α16}-hCCR5 cells were then resuspended (1×10⁶/mL) in ice cold binding buffer (50 mM HEPES, 1 mM CaCl₂, 5 mM MgCl₂, 0.5% BSA, 0.05% NaN₃, pH 7.24), pH 7.4), supplemented with freshly made 0.5% BSA and 0.05% NaN₃.

Eighty μl CHO-G_{α16}-hCCR5 (1×10⁶/mL) cells were added to 96 well plates. All dilutions were made in binding buffer (50 mM HEPES, 1 mM CaCl₂, 5 mM MgCl₂, 0.5% BSA, 0.05% NaN₃, pH 7.24).

The plates were incubated on a cell shaker at RT for 2 h with a final concentration of 0.1 nM ¹²⁵I RANTES or ¹²⁵I MIP-1α or ¹²⁵I MIP-1β. The compound dilutions were made in PBS, 1% BSA. Total reaction volume was 100 μl per well. The test compounds were added to the cells prior to the addition of radioligand.

After incubation, the cells were harvested onto GF/C filter plates using Packard cell harvester. Filters were pretreated with 0.3% PEI/0.2% BSA for 30 min. The filter plate was washed rapidly 5 times with 25 mM HEPES, 500 mM NaCl, 1 mM CaCl₂ and 5 mM MgCl₂ adjusted to pH 7.1. Plates were dried in oven (70° C.) for 20 min, added with 40 μl scintillation fluid and sealed with Packard TopSeal-A. Packard Top Count was used to measure of the radioactivity for 1 min per well.

Total binding was determined with control wells added with radioisotope and buffer and the non-specific binding was determined using an excess cold RANTES to some of the control wells. Specific binding was determined by subtracting the non-specific form total binding. Results are expressed as the percentage of specific ¹²⁵I RANTES binding. IC₅₀ values were determined using varying concentrations of the test ligand in triplicates and the data was analyzed using GraphPad Prism (GraphPad, San Diego, Calif.).

### Example 7

### CCR5-Mediated CCF Assay

CCF assay was performed as described before (C. Ji, J. Zhang, N. Cammack and S. Sankuratri, J. Biomol. Screen. 2006 11(6):652-663). Hela-R5 cells (express gp160 from R5-tropic virus and HIV-1 Tat) were plated in 384 well white culture plates (BD Bioscience, Palo Alto, Calif.) at 7.5×10³ cells per well in phenol red-free Dulbecco's Modified Eagle Medium (DMEM) supplemented with 10% FBS, 1× Pen-Strep, 300 μg/mL G418, 100 μg/mL hygromycin, and 1 μg/mL Doxycycline (Dox) (BD Bioscience, Palo Alto, Calif.), using Multimek (Beckman, Fullerton, Calif.) and incubated at 37° C. overnight to induce the expression of gp160. Ten μL diluted compounds in medium containing 5% DMSO were added to the cells, followed by the addition of CEM-NKr-CCR5-Luc (obtained from NIH AIDS Research & Reference Reagents Program) that expresses CD4 and CCR5 and carries a HIV-2 long terminal repeat (LTR)-driven luciferase reporter gene at 1.5×10⁴ cells/15 μL/well and incubated for 24 hrs. At the end of co-culture, 15 μL of Steady-Glo luciferase substrate was added into each well, and the cultures were sealed and gently shaken for 45 min. The luciferase activity were measured for 10 sec per well as luminescence by using 16-channel TopCount NXT (PerkinElmer, Shelton, Conn.) with 10 min dark adaptation and the readout is count per second (CPS). For the drug interaction experiments, small molecule compounds or antibodies were serially diluted in serum-free and phenol red-free RPMI containing 5% DMSO (CalBiochem, La Jolla, Calif.) and 1× Pen-Strep. Five μL each of the two diluted compound or mAb to be tested for drug-drug interactions were added to the Hela-R5 cells right before the addition of target cells.

TABLE II

| | Cell-Cell Fusion (CCF) Assay |
|---|---|
| Cpd. No. | IC₅₀ (μM) |
| I-1 | <0.0025 |

### Example 8

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation (F)

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy | 0.01 |
| anisole) | |
| Water | q.s. 100 |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A compound according to formula I 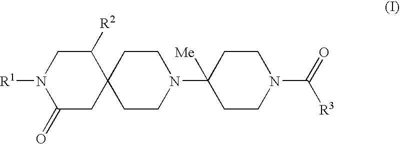
wherein:
R¹ is tetrahydropyranyl-methyl, tetrahydrofuranyl-methyl, 4-C₁₋₆ alkoxy-cyclohexylmethyl, 4-hydroxy-cyclohexylmethyl, C₃₋₆ cycloalkyl-C₁₋₃ alkyl, or IIa to IId 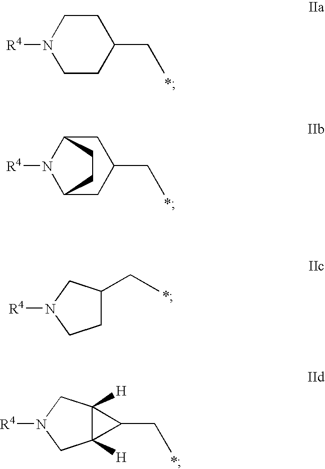
wherein:
R⁴ is —C(═O)OR⁵, —SO₂R⁵, C₁₋₆ acyl, C₁₋₆ haloalkyl;
said cycloalkyl is optionally independently substituted with one to three groups independently selected from the group consisting of hydroxy, C₁₋₆ alkoxy, C₁₋₃ alkyl, oxo, and halogen;
R² is C₁₋₆ alkyl, C₁₋₆ alkenyl, or C₁₋₄ alkoxy-C₁₋₃ alkyl;
R³ is selected from the group consisting of (a)-(e) and (f):
(a) 4,6-dimethyl-pyrimidin-5-yl;
(b) 4,6-dimethyl-2-trifluoromethyl-pyrimidin-5-yl;
(c) 2,4-dimethyl-pyridin-3-yl;
(d) 6-cyano-2,4-dimethyl-pridin-3-yl;
(e) 2,4-dimethyl-6-oxo-1,6-dihydro-pyridin-3-yl;
(f) 1,2,4-trimethyl-6-oxo-1,6-dihydro-pyridin-3-yl;
R⁵ is C₁₋₆ alkyl; or
mixtures of diastereomers, enantiomers or purified enantiomers or pharmaceutically acceptable salts thereof.

2. A compound according to claim 1 wherein:
R¹ is cyclohexyl optionally substituted by C₁₋₆ alkoxy;
R² is n-Bu;
R³ is (a), (c) or (d).

3. A compound according to claim 1 wherein:
R¹ is tetrahydropyranyl-methyl or tetrahydrofuranyl-methyl;
R² is n-Bu;
R³ is (a), (c) or (d).

4. A compound according to claim 3 wherein R¹ is tetrahydropyran-4-yl-methyl.

5. A compound according to claim 3 wherein R¹ is tetrahydrofiran-3-yl-methyl.

6. A compound according to claim 1 wherein:
R¹ is IIa;
R² is n-Bu;
R³ is (a), (c) or (d); and
R⁴ is C(═O)OR⁵, —SO₂R⁵ or C₁₋₆ acyl.

7. A compound according to claim 1 selected from the group consisting of:
5-Butyl-9-[1-(4,6-dimethyl-pyrimidine-5-carbonyl)-4-methyl-piperidin-4-yl]-3-(4-ethoxy-cyclohexylmethyl)-3,9-diaza-spiro[5.5]undecan-2-one;
5-Butyl-9-[1-(4,6-dimethyl-pyrimidine-5-carbonyl)-4-methyl-piperidin-4-yl]-3-(tetrahydro-pyran-4-ylmethyl)-3,9-diaza-spiro[5.5]undecan-2-one;
5-Butyl-9-[1-(4,6-dimethyl-pyrimidine-5-carbonyl)-4-methyl-piperidin-4-yl]-3-(4-methoxy-cyclohexylmethyl)-3,9-diaza-spiro[5.5]undecan-2-one;
5-Butyl-9-[1-(4,6-dimethyl-pyrimidine-5-carbonyl)-4-methyl-piperidin-4-yl]-3-(1-methanesulfonyl-piperidin-4-ylmethyl)-3,9-diaza-spiro[5.5]undecan-2-one;
3-(1-Acetyl-piperidin-4-ylmethyl)-5-butyl-9-[1-(4,6-dimethyl-pyrimidine-5-carbonyl)-4-methyl-piperidin-4-yl]-3,9-diaza-spiro[5.5]undecan-2-one;
4-{1-Butyl-9-[1-(4,6-dimethyl-pyrimidine-5-carbonyl)-4-methyl-piperidin-4-yl]-4-oxo-3,9-diaza-spiro[5.5]undec-3-ylmethyl}-piperidine-1-carboxylic acid methyl ester;
5-Butyl-9-[1-(4,6-dimethyl-pyrimidine-5-carbonyl)-4-methyl-piperidin-4-yl]-3-(4-ethoxy-cyclohexylmethyl)-3,9-diaza-spiro[5.5]undecan-2-one;
5-Butyl-9-[1-(2,4-dimethyl-pyridine-3-carbonyl)-4-methyl-piperidin-4-yl]-3-[(S)-1-(tetrahydro-furan-3-yl)methyl]-3,9-diaza-spiro[5.5]undecan-2-one;
5-Butyl-9-[1-(4,6-dimethyl-pyrimidine-5-carbonyl)-4-methyl-piperidin-4-y]-3-(tetrahydro-pyran-2-ylmethyl)-3,9-diaza-spiro[5.5]undecan-2-one;
5-Butyl-9-[1-(2,4-dimethyl-pyridine-3-carbonyl)-4-methyl-piperidin-4-yl]-3-(tetrahydro-pyran-4-ylmethyl)-3,9-diaza-spiro[5.5]undecan-2-one;
5-Butyl-9-[1-(4,6-dimethyl-pyrimidine-5-carbonyl)-4-methyl-piperidin-4-yl]-3-(tetrahydro-pyran-4-ylmethyl)-3,9-diaza-spiro[5.5]undecan-2-one;
5-Butyl-9-[1-(4,6-dimethyl-pyrimidine-5-carbonyl)-4-methyl-piperidin-4-yl]-3-(tetrahydro-pyran-4-ylmethyl)-3,9-diaza-spiro[5.5]undecan-2-one;
5-{4-[7-Butyl-10-oxo-9-(tetrahydro-pyran-4-ylmethyl)-3,9-diaza-spiro[5.5]undec-3-yl]-4-methyl-piperidine-1-carbonyl}-4,6-dimethyl-pyridine-2-carbonitrile;
5-Butyl-9-[1-(2,4-dimethyl-pyridine-3-carbonyl)-4-methyl-piperidin-4-yl]-3-[(R)-1-(tetrahydro-furan-3-yl)methyl]-3,9-diaza-spiro[5.5]undecan-2-one;
5-Butyl-9-[1-(2,4-dimethyl-pyridine-3-carbonyl)-4-methyl-piperidin-4-yl]-3-(4-hydroxy-cyclohexylmethyl)-3,9-diaza-spiro[5.5]undecan-2-one; or,
mixtures of diastereomers, enantiomers or purified enantiomers or pharmaceutically acceptable salts thereof.

8. A method for treating a human immunodeficiency virus (HIV-1) infection, or treating AIDS or ARC, in a patient in need thereof which comprises administering to the patient in need thereof a therapeutically effective amount of a compound according to claim 1.

9. A method according to claim 8 further comprising co-administering a therapeutically effective amount of one or more compound(s) selected from the group consisting of HIV-1 nucleoside reverse transcriptase inhibitors, non-nucleoside reverse transcriptase inhibitors, HIV-1 protease inhibitors, HIV-1 integrase inhibitors and HIV-1 viral fusion inhibitors and a compound of claim 1.

10. A method for treating rheumatoid arthritis in a patient in need thereof comprising administering a compound according to claim 1.

11. A method according to claim 10 further comprising co-administering a therapeutically effective amount of one or more anti-inflammatory or analgesic compounds and a compound of claim 1.

12. A method for treating asthma or congestive obstructive pulmonary disease (COPD) comprising administering a therapeutically effective amount of a compound according to claim 1 to a patient in need thereof.

13. A method for treating solid organ transplant rejection comprising administering a therapeutically effective amount of a compound according to claim 1 to a patient in need.

14. A method according to claim 13 further comprising co-administering a therapeutically effective amount of one or more anti-rejection drugs or immunomodulators and a compound of claim 1.

15. A pharmaceutical composition comprising a compound according to claim 1 and at least one pharmaceutically acceptable carrier, diluent or excipient.
